(19) United States
(12) Plant Patent Application Publication
(10) Pub. No.: US 2006/0225170 P1
(43) Pub. Date: Oct. 5, 2006

(54) Symphoricarpos plant named 'Brain De Soleil'

(76) Inventor: Pascal Quemard, Brain sur l'Authion (FR)

Correspondence Address: BUCHANAN, INGERSOLL & ROONEY PC, POST OFFICE BOX 1404, ALEXANDRIA, VA, 22313-1404, US

(21) Appl. No.: 11/392,492

(22) Filed: Mar. 30, 2006

(30) Foreign Application Priority Data
Mar. 31, 2005 (QZ) 2005/0625

(51) Int. Cl. A01H 5/00

(52) U.S. Cl. PLT/263

(57) ABSTRACT

A new and distinct variety of Symphoricarpos×Chenaultii plant, commonly known as Chenault Coralberry, is provided. The new variety originated as a naturally occurring mutation of the ‘Hancock’ variety (non-patented in the United States) at Maine et Loire, France. Unlike the parental variety, the new variety forms attractive yellow-orange foliage. The new variety is low-growing and displays a ground cover growth habit. The distinctive combination of characteristics renders the new variety to be well suited for growing as attractive ornamentation in the landscape.

### BOTANICAL/COMMERCIAL CLASSIFICATION

Symphoricarpos×Chenaultii/Chenault Coralberry

### VARIETAL DENOMINATION

cv. ‘Brain De Soleil’

### SUMMARY OF THE INVENTION

A new variety of Symphoricarpos plant, botanically known as Symphoricarpos×Chenaultii is provided.

The new variety of the present invention was discovered during 2003 at Maine et Loire, France, while growing among plants of the ‘Hancock’ variety (non-patented in the United States). The new variety is a natural mutation of the ‘Hancock’ variety of unknown causation. I was primarily attracted to the plant of the new variety in view of it is distinctive foliage coloration. Had I not discovered and preserved the plant of the new variety it would have been lost to mankind.

I was found that the plant of the new variety exhibits the following combination of characteristics:
(a) displays attractive yellow-orange foliage unlike the green foliage of its parent ‘Hancock’ variety (non-patented in the United States),
(b) displays a ground cover growth habit, and
(c) is well suited for growing as attractive ornamentation in the landscape.

The new variety of the present invention readily can be distinguished from other Symphoricarpos×Chenaultii varieties, including its parent of the ‘Hancock’ variety in view of its distinctive foliage coloration.

The new variety well meets the needs of the horticultural industry and is particularly well suited for providing attractive ornamentation in the landscape.

The new variety of the present invention has been found at Maine et Loire, France, to undergo asexual propagation by the rooting of cuttings. The distinctive combination of characteristics of the new variety is reliably transmitted to subsequent generations following such propagation. Accordingly, the new variety propagates in a true-to-type manner via asexual propagation.

The new variety has been named ‘Brain De Soleil’.

### BRIEF DESCRIPTION OF THE PHOTOGRAPH

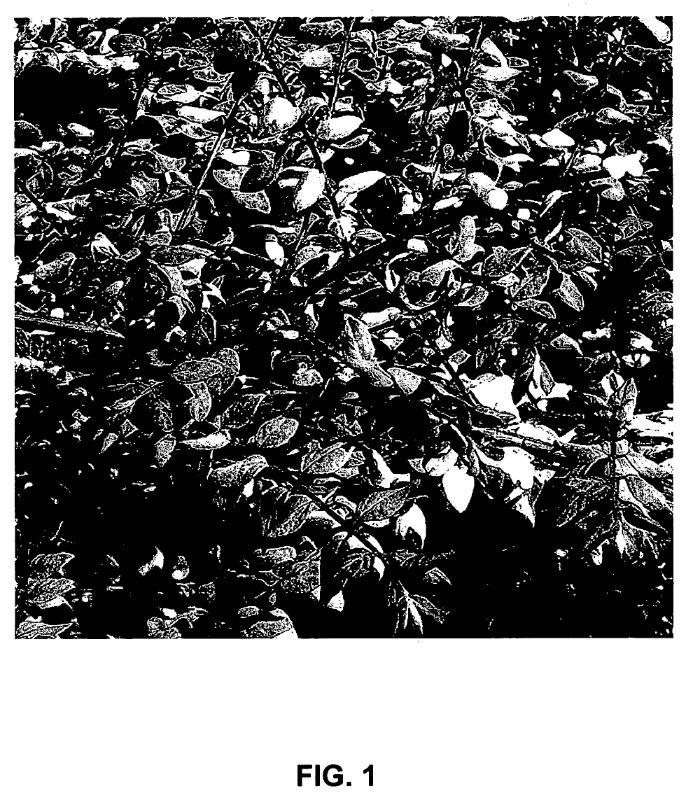

The accompanying photograph of FIG. 1 depicts the attractive yellow-orange foliage and red-brown branches of a typical plant of the new variety. Such plant was approximately two years of age and was growing in the landscape during the summer at Maine et Loire, France.

### DETAILED DESCRIPTION

The following description is based on the observation of a two-year-old specimen of the new variety growing outdoors at Maine et Loire, France. Color designations are with reference to the R.H.S. Colour Chart of The Royal Horticultural Society, London, England. When employed, common color terms are to be accorded their customary dictionary significance.
Origin: A natural mutation of the ‘Hancock’ variety (non-patented in the United States) of unknown causation.
Plant:
   Habit.—spreading as a ground cover.
   Height.—commonly approximately 50 to 60 cm on average.
   Width.—commonly approximately 100 to 150 cm on average.
Branches:
   Color.—red-brown.
Foliage:
   General appearance.—the foliage is deciduous, shoots change from yellow to orange in coloration at full maturity, and the brightness varies somewhat with the season.
   Leaf size.—length: commonly approximately 0.6 to 1 cm on average. — width: commonly approximately 0.4 to 0.8 cm on average.
   Leaf color.—Springtime: near Orange-Red Group 31A and 31B and Red Group 37A and 37B. Summer: near Yellow Group 10B and 10C. December: near Yellow-Orange Group 15B and Yellow-Orange Group 16A.
Inflorescence:
   Time of blooming.—May.
   Duration of blooming.—approximately one month depending upon environmental conditions.
   Size.—very small and inconspicuous.
   Quantity.—sparse.
   Hip size.—commonly 0.5 to 0.8 cm on average.
   Hip color.—near Red-Purple Group 61B and 61C.

During observations to date the resistance to disease has been excellent under the growing conditions of Maine et Loire, France.

Plants of new ‘Brain De Soleil’ variety have not been observed under all possible environmental conditions to date. Accordingly, it is possible that the phenotypic expression may vary somewhat with changes in light intensity and duration, cultural practices, and other environmental conditions.

What is claimed is:

1. A new and distinct Symphoricarpos×Chenaultii plant that exhibits the following combination of characteristics:
(a) displays attractive yellow-orange foliage unlike the green foliage of its parent ‘Hancock’ variety (non-patented in the United States),
(b) displays a ground cover growth habit, and
(c) is well suited for growing as attractive ornamentation in the landscape;
substantially as illustrated and described.
